(19) United States
(12) Patent Application Publication
(10) Pub. No.: US 2024/0423877 A1
(43) Pub. Date: Dec. 26, 2024

(54) LIGHT CURING AND SINTERABLE COMPOSITE, SYSTEMS AND METHODS FOR ADDING AESTHETIC FEATURES TO A DENTAL CERAMIC BASE

(71) Applicant: Harvest Dental Products, LLC, Brea, CA (US)

(72) Inventors: Sasha Der Avanessian, Cary, NC (US); Jorg Erxleben, Brea, CA (US); Zaza Gomurashvili, Tustin, CA (US)

(21) Appl. No.: 18/663,275

(22) Filed: May 14, 2024

Related U.S. Application Data
(60) Provisional application No. 63/521,894, filed on Jun. 20, 2023.

(51) Int. Cl. A61K 6/833; A61C 13/08; A61K 6/884

(52) U.S. Cl. CPC A61K 6/833; A61C 13/081; A61K 6/884

(57) ABSTRACT

A composite, three-dimensionally stable facing material for adding aesthetic features to a ceramic dental prosthetic base. The composite may be a malleable, pasty composite or flat strip of material capable of being molded to surfaces of the ceramic base and retained on the base. The applied material is capable of being trimmed, cured to a hardened state and finally sintered to its final hardness. Red or Violet ceramic fibers and/or other characterization may be added for increasing realism of a ceramic denture gingival surfaces.

### CROSS REFERENCE TO RELATED APPLICATIONS

This application claims the benefit of U.S. Provisional Patent Application Ser. No. 63/521,894, filed on Jun. 20, 2023, the disclosure of which is incorporated herein by reference in its entirety. This application is also generally related to the disclosure in U.S. patent application Ser. No. 18/128,455, filed on Mar. 30, 2023 (pending), the disclosure of which is incorporated by reference herein.

### TECHNICAL FIELD

The present invention generally relates to ceramic dental prosthetics, such as full and partial dentures and, more particularly, to manners of adding aesthetic features to a ceramic denture or other dental prosthetic to facilitate a more natural appearance and durable result.

### BACKGROUND

The growth in usage of zirconia or other ceramics for partial and/or full dentures in the last years has created esthetic challenges for the dental technicians. A dental prosthesis should harmonize with the soft tissues that surround the dentition, especially in the anterior region. This desire is primarily so that the prosthetic is as undetectable as possible during use by the wearer. Ceramic materials such as zirconia have proven to be very successful base materials for implant supported dentures. But, on the other hand, conventional ceramic denture or other prosthetic materials offer more limited esthetics because they do not have a natural appearance. For example, in the case of dentures, a healthy-looking gingiva is the result of the combination of a bone sub-structure covered by a somewhat transparent, pinkish gingival tissue with veins. To achieve this esthetic goal, the technician masks the already-sintered ceramic base typically with light curing composites or with burn-on ceramic paints. The light curing composites present lower overall bond strengths to the ceramics and are susceptible to color changes during usage. Burn-on ceramic materials, on the other hand, have excellent strength and bonding characteristics but are very time-consuming in application and the final shade is only revealed after sintering. This procedure is difficult and currently only executed by very experienced technicians. The additional sintering may also weaken the substructure of tetragonal zirconia. All of these techniques are highly dependent on the artistic abilities of the technician and are very time-consuming.

Various techniques for gingival surface characterization of a ceramic restoration are available to a dental technician, although industry would find advantage in new methods and materials which would simplify this process and provide more durable and consistent results. It would therefore be desirable to provide a light-curing sinterable composite-ceramic system, including auxiliaries for achieving a highly durable, authentic and realistic gingival surface for a ceramic denture that is less time-consuming and less dependent on specialized technician artistry or skills.

### SUMMARY

Generally, in one aspect or option, a semi burn-out meltable (sinterable) facing material is provided for adding texture and color to the gingival area of a ceramic denture base structure. The ceramic material may be zirconia or another ceramic material. This may take the form of a three-dimensionally stable, formable facing material applied to a dentine/bone-like substructure of the ceramic. To the facing material the user may add texture and anatomical features such as shade variation/characterization and replicas of gingival veins. Individual staining may be drawn with a very fine brush onto the outside surface of the facing material or underneath the facing material. Bone-like substructures, dark reddish and whitish areas as well as any other features, may be added to make the denture appear more life-like. All of these features may be added using the facing material itself and/or provided liquid light-curing modifiers (e.g., stains) for enhanced characterization replicating the natural gingiva. The facing material will be easily applied to and hand worked on the pre-sintered or already-sintered gingival surface of the ceramic denture. After the final characterization to achieve a more life-like appearance, the facing material is pre-cured with visible and/or UV light to a final shape and characterization for esthetic evaluation before sintering. The characterization may be further adjusted as necessary.

After pre-curing and acceptable evaluation of the characterization, the material is sintered onto the ceramic denture base to a final hardness and appearance. During the sintering process the polymerized organic constituents of the facing material burn out without leaving any residues. After sintering the facing material is polished/finished using the usual methods of finishing ceramics.

In an illustrative embodiment, the invention provides a composite facing material for adding aesthetic features to a ceramic dental prosthetic base. The composite provides a flexible material capable of being molded to prosthetic surfaces, such as gingival surfaces of a denture base, and retained on the base, the facing material further being capable of being trimmed and then cured to a semi-hardened state and finally sintered to a hardened ceramic state. Various characterization pastes, fluids and powder/liquid systems are provided. As examples, the facing material may be delivered as a paste, flowable paste or liquid, a cylindrical “rope”, a flat sheet, formed as a flat strip or rolled forms of flat material such as a roll of tape.

The facing material is formed at least in part from a polymerizable oligomeric material. The oligomeric material stabilizes the ceramic on the surface during modeling, approval and sintering. Other (Meth-) acrylates are added to control polymerization and viscosity of the final product. Specific examples for the material are provided below in the Example section, but general examples of oligomeric material are:
Oligo/Poly-Urethane Di-(Tri) Methacrylates Viscosity 4,000-20,000 cp (CAS #72869-86-4)
Oligo/Poly-Alkyleneglycol Di-(Tri) Methacrylates Viscosity 4,000-20,000 cp (CAS #25852-47-5)
Oligo/Poly-Epoxy Di-(Tri) (Meth-)acrylates Viscosity 4,000-20,000 cp (CAS #71281-65-7)
with a wide molecular weight and viscosity range. These monomers may be used in any possible combination.

The facing material is formed at least in part from a vitreous material. The vitreous material (Fluor/Sodium/Magnesium Silicate Glasses) forms at high temperatures (1000° C.-1500° C.) together with the pyrogenic silica and the melt modifiers a homogeneous high viscosity melt. This maintains the initially given characterization. A specific example for the material is provided below in the Example section, but general examples of a vitreous material are:
Fluor-/Sodium-/Magnesium-Silicate Glasses (CAS #7440-2-4)
Pyrogenic Silica (CAS #112945-52-5)
Melt viscosity controlling Aluminum-, Calcium-, Magnesium-, Boron-, etc., salts.
with a wide grain size and melting point range.

The facing material may be delivered as powder/liquid system with a liquid containing:
Oligo/Poly-Alkyleneglycol Di-(Tri) Methacrylates
Viscosity 4,000-20,000 cp (CAS #25852-47-5) 40.0% 70.0%
(Meth-) acrylate Monomers, and 29.5%-59.5%
polymerization-initiators, 0.5%-2.0%
   and powder containing:
(co-) initiators, 0.5%-2.0%
pyrogenic silicas, 10.0%-20.0%
Aluminum-, Calcium-, Magnesium-,
Boron-, etc., salts. 0.2%-10.0%
ceramic pigments and, 0.2%-20.0%
low melting glass-ceramic 50.0%-80.0%
   which are mixed 2 parts of powder into the liquid, then applied to the ceramic surface and polymerized.
   Example 1 shows a suitable formulation where powder and liquid are dosed 2:1, mixed and applied to the ceramic surface and polymerized. Eventually, pigments may be added to the powder before adding to the liquid.

The facing material may further be delivered as a polymerizable paste, flowable paste or liquid containing:
Oligo/Poly-Epoxy Di-(Tri) (Meth-)acrylates
Viscosity 4,000-20,000 cp (CAS #71281-65-7) 20.0%-35.0%
(Meth-) acrylate Monomers, and 15.0%-30.0%
polymerization-initiators, 0.2%-1.0%
(co-) initiators, 0.2%-1.0%
pyrogenic silicas, 5.0%-10.0%
Aluminum-, Calcium-, Magnesium-,
Boron-, etc., salts. 0.1%-5.0%
ceramic pigments and, 0.1%-10.0%
low melting glass-ceramic. 40.0%-70.0%
   which are applied to the ceramic surface and photo-polymerized.
   Example 2 shows a suitable formulation which is applied to the zirconia/ceramic surface and polymerized.

The facing material is preferably manufactured preformed such as a cylindrical “rope”, a flat sheet, formed as a flat strip or rolled forms of flat material such as a roll of tape from a combination of
(Meth-) acrylate Monomers, 15.0%-30.0%
Oligo/Poly-Urethane Di-(Tri) Methacrylates
Viscosity 4,000-20,000 cp (CAS #72869-86-4) 20.0%-35.0%
polymerization-initiators, 0.2%-1.0%
(co-) initiators, 0.2%-1.0%
pyrogenic silicas, 5.0%-10.0%
Aluminum-, Calcium-, Magnesium-,
Boron-, etc., salts. 0.1%-5.0%
ceramic pigments and, 0.1%-10.0%
low melting glass-ceramic. 40.0%-70.0%
   which are applied to the zirconia/ceramic surface and photo-polymerized.
   Example 3 shows a suitable formulation which is applied to the ceramic surface and polymerized.

The facing material is mechanically and/or chemically bonded to the ceramic denture or other prosthetic dental base. The facing material may be light-curable and may be at least partially translucent. The packaging should maintain the facing material being protected from light and mechanical damage. Other packaging precautions (such as protection from air, particulate contamination, heat, etc.) may be taken based on the needs of the material. The facing material may be textured on its surface(s) to resemble gingival tissues during manufacture, if desired, or other natural tissues or surfaces.

In another illustrative aspect, a ceramic dental prosthetic is provided and includes a base, a malleable facing material molded to the prosthetic surfaces of the ceramic base and retained on the base to provide the look of natural surfaces, such as gingival surfaces before being sintered. The facing material may further include ceramic fibers for replicating the vein structure of gingival tissue and may include liquid modifiers to provide enhanced coloring to the facing material.

In another illustrative aspect, the invention further provides a method of adding aesthetic features to a ceramic denture base. The method includes applying a malleable facing material to surfaces of the prosthetic dental appliance base, trimming the facing material to conform to the surfaces of the base and sintering the facing material into a hardened condition. The facing material may be formed at least in part from a vitreous material. The method may further include using light-curable liquid stains to provide characterization to the facing material.

In another illustrative aspect, the invention further provides a method of adding aesthetic features to a ceramic base of a prosthetic dental appliance, such as a denture or other prosthetic. The method includes the feature to verify the esthetical aspect before sintering in vitro. This way, esthetical corrections may be made before final sintering of the prosthetic.

Additional features and advantages of the inventive aspects will become more apparent upon review of the following detailed description taken together with accompanying drawings of the illustrative and exemplary embodiments.

### BRIEF DESCRIPTION OF DRAWINGS

FIG. 1 is a top perspective view of a flexible, malleable aesthetic strip of facing material about to be applied to a denture base.

FIG. 1A is a side elevational view of the denture base shown in FIG. 1, with the flexible, malleable strip of facing material schematically shown in dash-dot lines just prior to being applied and molded to the denture base.

FIG. 2 is a top perspective view of the flexible, malleable aesthetic strip of facing material being applied to the denture base.

FIG. 3 is a top perspective view of the flexible, malleable aesthetic strip of facing material being molded by hand to the denture base.

FIG. 4 is a top perspective view of the flexible, malleable aesthetic strip of facing material after being molded and adhered to the denture base, and undergoing a trimming process.

FIG. 4A is a side elevational view of the flexible, malleable aesthetic strip of facing material after being molded and adhered to the denture base, and undergoing a trimming process as illustrated in FIG. 4.

FIG. 5 is a top perspective view of the denture base and the molded, adhered and trimmed strip of facing material being cured into a hardened condition.

FIG. 6 is a top perspective view of the denture base and the molded, adhered, trimmed and cured strip of facing material being polished.

FIG. 6A is a side elevational view of the denture shown in FIG. 6 being polished.

FIG. 7 is a top elevational view of the denture shown in FIGS. 6 and 6A being colored, such as with a stain applied with a brush.

FIG. 8 is a perspective view of a strip of facing material constructed in accordance with an illustrative embodiment in a flat, rectangular form.

FIG. 9 is a perspective view of a strip of facing material constructed in accordance with an illustrative embodiment in a roll form.

FIG. 10 is an elevation view of the facing material in a flat, sheet form.

### DETAILED DESCRIPTION

An illustrative embodiment of the invention relates to a malleable, biocompatible dental facing material that has sufficient strength to maintain its shape as a three-dimensionally stable material during transportation and storage yet has sufficient malleability to be subsequently formed or molded, such as by hand, into a final shape on the gingival surfaces of the ceramic denture base, then pre-cured to a hardened state for esthetic evaluation, and then sintered to a final hardness and polished as needed. This material may be used as a veneer to improve the esthetics of a gingival surface portion of a removable or fixed ceramic dental prosthetic or denture. In other aspects, the surface portion may be any other surface of a dental prosthetic other than a gingival surface. The facing material is preferably comprised of a curable composite formed at least partially of a polymeric material and a partially meltable glass-ceramic/silica mix. The facing material may be delivered as a paste or liquid, a cylindrical “rope”, a flat sheet, formed as a flat strip or rolled forms of flat material such as a roll of tape. The material may be pre-cured with visible light, UV light, heat, or in any other manner depending on the material composition. Then the facing material can finally be sintered onto the ceramic substructure.

The uncured facing material is sufficiently strong, prior to pre-curing, that the three-dimensionally stable pre-formed shape and its features, such as malleability, workability, color retention and curability, are retained prior to pre-curing and sintering. This means that the facing material remains dimensionally stable while contained in its packaging for a sufficient time (e.g., one year at about 23° C. or room temperature) as well as during temporary spikes in temperature up to about 40° C. and down to about 10° C. The facing material will be sufficiently malleable prior to pre-curing for purposes of allowing final features to be formed, such as with easy hand molding by a dental technician. Preferably, the facing material is easily formed or worked by hand under a moderate force, using fingers and/or typical dental hand tools.

The composition of the facing material will facilitate its use as a ceramic gingival veneer, being generally a mixture of light-curing monomers, pyrogenic silica and fine glass-ceramics with a lowered melting point. During the controlled sintering process, the previously light-cured polymers will burn out and in parallel the lower melting point glass-ceramic will melt and incorporate the pyrogenic silica as a viscosity regulator. This way the melted glass-ceramic will be transformed into a “putty-like” state and remain at the application spot.

The facing material may be pre-formed during manufacture with coloring in various shades of pink to red, which are selected for the shade most similar to the natural gingiva of a particular patient. The coloring may be partially translucent, translucent and/or opaque. The area on the denture base to receive the facing material is cleaned, then the facing material is applied onto the desired location. Additional trimming and forming is completed as needed (such as at the gingival line and interdental papilla) to mimic the natural gingiva. Provided modifiers may be used to enhance the life-like characterization of the facing material, and once the technician is pleased with the esthetics of the gingival portion of the denture, the facing material is pre-cured to a temporary hardness. The prosthetic or denture (or pictures of it) may then be sent to the treating dentist for esthetic evaluation. After approval, the prosthetic or denture is sintered, polished as needed and delivered for patient use.

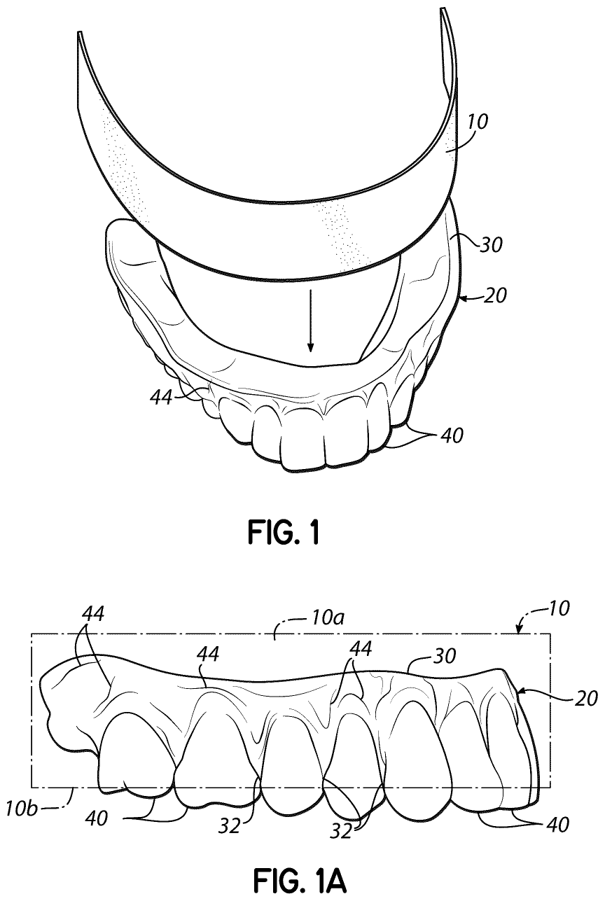

Referring to the example of a denture illustrated in FIGS. 1 and 1A, the facing material 10 is positioned relative to a denture base 20 such that the upper portion 10a of the material ends at the upper gingival portions 30 of the denture base 20 and a lower edge 10b of the material 10 extends below the lowermost points 32 of the gingiva between the prosthetic teeth 40. Veins may be simulated by adding suitable fibers 44. These fibers 44 may be adhered to the denture base 20, as shown, and/or incorporated into or onto the material 10 as desired for enhancing realism.

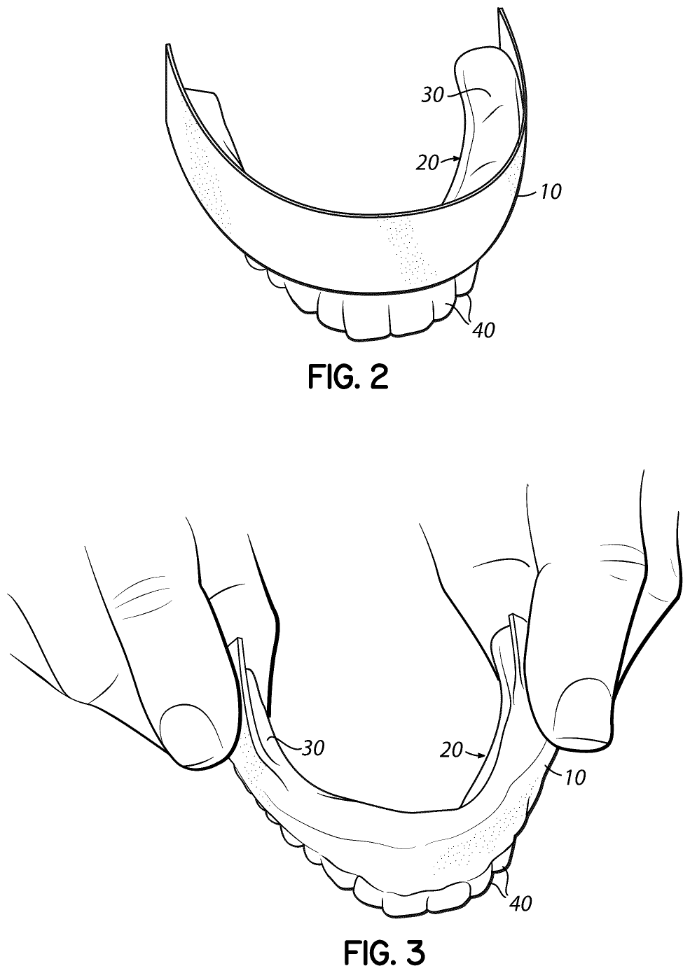

As further shown in FIGS. 2 and 3, the material 10 may be hand molded or similarly molded by hand instrument by pressing the material 10 against the prosthetic gingival surfaces adjacent to the teeth 40 and upper gingival portions 30 as particularly shown in FIG. 3.

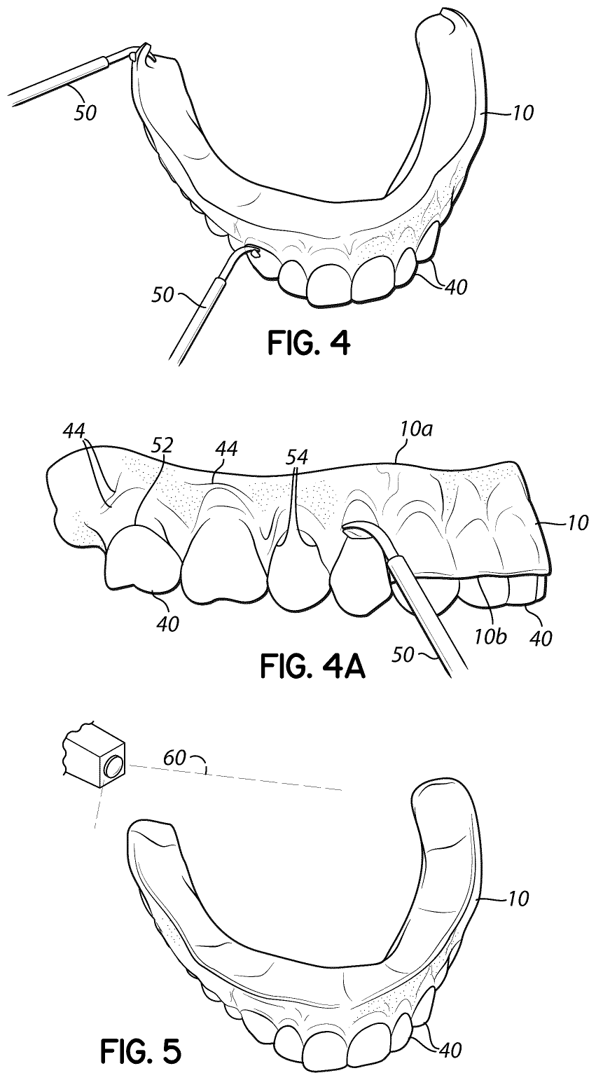

As shown in FIGS. 4 and 4A, dental hand tools 50 are used to appropriately trim the material 10 to create realistic gingival lines 52 and papilla lines 54 adjacent the prosthetic teeth 40.

As further shown schematically in FIG. 5, light 60 is used to pre-cure the molded and trimmed material 10 after the technician is satisfied with the esthetics, such that the material 10 is pre-hardened. The light 60 may be of any wavelength necessary to cure the light-curable material formulation used for the material 10. Preferably, the light 60 is in the visible portion of the spectrum.

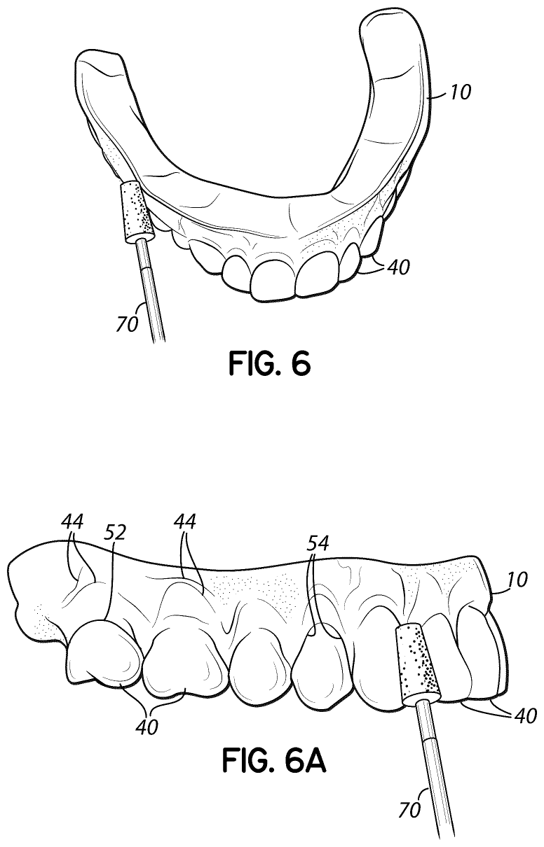

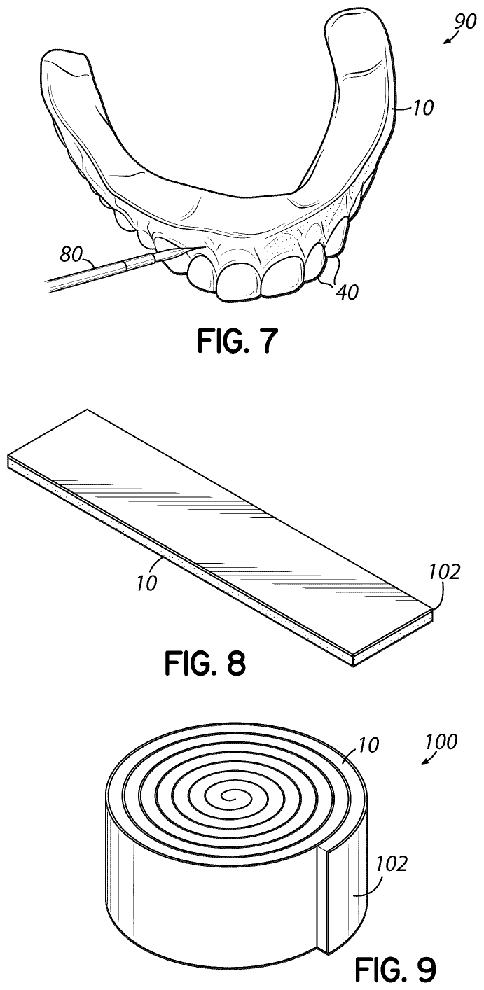

As further shown in FIGS. 6 and 6A, the pre-hardened material 10 is then polished by one or more tools 70 as needed or desired by the technician to achieve the desired look. As further shown in FIG. 7, characterization may be added by the technician, such as by using an application brush 80 and/or other tool brushing stain or otherwise applying color to the visible surfaces of the prosthetic gingiva comprised of the material 10. Other manners of adding life-like characterization may be used depending on the desires of the technician.

At this stage the ready-to-sinter denture is in the stage to be approved by the dentist for final sintering. If corrections are needed, these are easy to realize by usual technical methods.

After approval by the dentist, the whole denture is sintered to achieve the final denture. The denture 90 is then complete and may be fitted to the patient.

FIGS. 8 and 9 illustrate two possible delivery forms of material, including a rectangular strip 10 shown in FIG. 8 and another form of the material 10 that has been placed into a rolled condition 100, similar to a roll of tape, shown in FIG. 9. Each of these forms may include a release film or paper 102 which is peeled off to expose an adhesive side of the material 10.

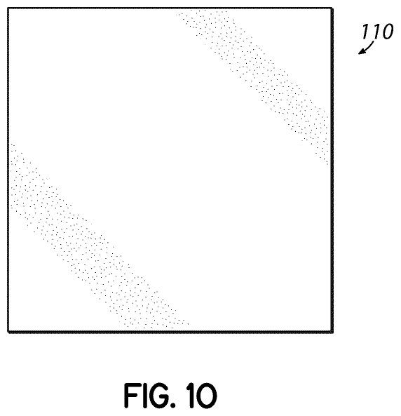

FIG. 10 illustrates a sheet 110 of the facing material. Sheet 110 may have any desired shape and/or dimensions from which smaller and more narrow strips of the facing material may be cut or otherwise removed or separated for use. As one of many possible examples, the sheet may be 200 mm(l)×200 mm(w)×2.5 mm(t). Of course, any other size may be used instead.

### Example 1

An illustrative formulation of the facing material delivered as powder/liquid system where the liquid is comprised of:
68.00% PEG200DMA (CAS #25852-47-5)
12.35% TMPTMA (Trimethylolpropane Trimethacrylate (CAS #3290-92-4)
19.35% Urethane Methacrylate Viscosity 8,000 cp (CAS #72869-86-4)
0.09% 2,4,6-trimethylbenzoyldiphenyl phosphine oxide (CAS #162881-26-7)
0.09% Camphor quinone (CAS #10373-78-1)
0.08% EDMAB (CAS #10287-53-3)
0.04% 3,5-Di-tert-4-butylhydroxytoluene (CAS #128-37-0),
   and, where the powder is comprised of:
80.00% Low Melting (350°° C.-600° C.) Glass Frit (CAS #65997-18-4)
5.00% Aluminum Hydroxide (CAS #21645-51-2)
5.00% Sodium Borate (CAS #1303-96-4)
8.00% Pyrogenic Silica (200m²/g) (CAS #112945-52-5)
2.00% Ceramic Pigments (White, Red, Blue and Yellow)
   Powder and liquid are dosed 2:1, mixed and applied to the zirconia/ceramic surface and polymerized. Eventually needed pigments may be added to the powder before adding it to the liquid.
   This formulation is preferably used for additions and corrections on already sintered and veneered bridges and overdentures for corrections and additions. During the application, in appropriated stages, the material is precured under an approximation light. After finalizing the application, the material is polymerized for 5 min. in a 405 nm light-box.
   After approval by the responsible part, the restoration is burned in a ceramic furnace for 60 min.-120 min. at 600° C.-650° C. An appropriate heating rate is 30° C.-70° C./min.

### Example 2

Another illustrative formulation of the flowable facing material is comprised of:
34.00% Oligo Epoxy Di-Methacrylate Viscosity 12,000 cp (CAS #71281-65-7)
12.40% HEDMA (Hexamethylene di-methacrylate) (CAS #6606-59-3)
8.00% PEG600DMA (CAS #25852-47-5)
34.50% Low Melting (400° C.-800° C.) Glass Frit (CAS #7440-2-4)
8.00% Surface Treated Pyrogenic Silica (200 m²/g) (CAS #112945-52-5)
0.03% 2,4,6-trimethylbenzoyldiphenyl phosphine oxide (CAS #1628881-26-7)
0.03% Camphor quinone (CAS #10373-78-1)
0.02% EDMAB (CAS #10287-53-3)
0.02% 3,5-Di-tert-4-butylhydroxytoluene (CAS #128-37-0) 1.20%
3.00% Ceramic Pigments (White, Red, Blue and Yellow)
   The material is delivered as gel in syringes from which it is applied to the ceramic surface prior to the covering material to reproduce vein-or root-like appearance. This formulation is preferably used for characterizations underneath veneering dentures, bridges and overdentures. During the application, in appropriated stages, the material is precured under an approximation light. Then the material is covered by the main material and polymerized for 5 min. in a 405 nm light-box. After approval by the responsible part, the restoration is burned in a zirconia/ceramic furnace for 240 min.-420 min. between 800° C.-1,500° C. An appropriate heating rate is 50° C.-80° C./min.

### Example 3

The facing material is preferably manufactured preformed such as a cylindrical “rope”, a flat sheet, formed as a flat strip or rolled forms of flat material such as a roll of tape from a combination of
22.00% Urethane Methacrylate Viscosity 20,000 cp (CAS #72869-86-4)
12.40% TMPTMA (Trimethylolpropane Trimethacrylate (CAS #3290-92-4)
10.40% PEG200DMA (CAS #25852-47-5)
38.10% Low Melting (450° C.-800° C.) Glass Frit (CAS #65997-18-4)
15.00% Surface Treated Pyrogenic Silica (200 m²/g) (CAS #112945-52-5)
0.03% 2,4,6-trimethylbenzoyldiphenyl phosphine oxide (CAS #162881-26-7)
0.03% Camphor quinone (CAS #10373-78-1)
0.02% EDMAB (CAS #10287-53-3)
0.02% 3,5-Di-tert-4-butylhydroxytoluene (CAS #128-37-0)
3.00% Ceramic Pigments (White, Red, Blue and Yellow)
   This formulation is preferably used for veneering dentures, bridges and overdentures. During the application, in appropriated stages, the material is precured under an approximation light. Then the material is covered by the main material and polymerized for 5 min. in a 405 nm light-box.
   After approval by the responsible part, the restoration is burned in a zirconia/ceramic furnace for 240 min.-420 min. between 800° C.-1,500° C. An appropriate heating rate is 50° C.-80° C./min.

### Example 4

Another illustrative prosthetic tooth facing material is delivered in composite syringes and made from a combination of:
20.00% Urethane Methacrylate Viscosity 20,000 cp (CAS #72869-86-4)
10.60% TMPTMA (Trimethylolpropane Trimethacrylate (CAS #3290-92-4)
8.30% PEG200DMA (CAS #25852-47-5)
24.30% Low Melting (450° C.-800° C.) Glass Frit (CAS #65997-18-4)
23.70% High Melting (1,100° C.-1,300° C.) Glass Frit (Schott GM 27448)
10.00% Surface Treated Pyrogenic Silica (200 m²/g) (CAS #112945-52-5)
0.03% 2,4,6-trimethylbenzoyldiphenyl phosphine oxide (CAS #162881-26-7)
0.03% Camphor quinone (CAS #10373-78-1)
0.02% EDMAB (CAS #10287-53-3)
0.02% 3,5-Di-tert-4-butylhydroxytoluene (CAS #128-37-0)
3.00% Ceramic Pigments (White, Brown, Black and Yellow)
   This formulation is preferably tooth shaded and used for veneering full-dentures, bridges and overdentures. During the application, in appropriated stages, the material is precured under an approximation light. Then the material is covered by the main material and polymerized for 5 min. in a 405 nm light-box.
   After approval by the responsible part, the restoration is burned in a ceramic furnace for 120 min.-180 min. between 600° C.-900° C. under reduced pressure (vacuum). An appropriate heating rate is 40° C.-60° C./min.

The formulations present a depth of cure>4 mm and a good adhesivity to ceramics.

One illustrative strip size is: 70 mm(length)×10 mm(width)×2.5 mm (thickness).

The organic part of composition burns without any residuals above 300° C.

The lower melting glass and the pyrogenic silica form a jelly-like melt which consistency is adjustable to maintain the characterization in place.

A modifier stain may be formed using the same formulation as described in example 2 above, but with 25% less Pyrogenic Silica/Glass Frit in Red, Brown, Black, Blue, Yellow and Dark Pink. Special attention should be given to the possibility to characterize underneath the “protecting” overlayed composite facing material, which provides a protection to the esthetics of the facing material.

An illustrative and preferred bonding formulation applied to the denture base before applying the material is comprised of:
50.0% iso-Propanol (CAS #67-63-0)
15.0% Urethane Methacrylate Viscosity 8,000 cp (CAS #72869-86-4)
35.0% TMPTMA (Trimethylolpropane Trimethacrylate (CAS #3290-92-4)

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A composite for adding esthetic features to a ceramic prosthetic dental base, the composite comprising:
a flexible and three-dimensionally stable facing material capable of being molded to surfaces of a ceramic base and retained on the base, the facing material further being capable of being trimmed, then cured to a hardened state for pre-evaluation and then sintered into and/or onto the ceramic denture base.

2. The composite of claim 1, wherein the facing material is formed at least in part from a polymeric material.

3. The composite of claim 1, wherein the facing material is formed at least in part from a low melting glass-ceramic material.

4. The composite of claim 1, wherein the facing material is formed at least in part from a Pyrogenic Silica.

5. The composite of claim 3, wherein the low melting glass-ceramic material presents a melting temperature between 450° C. and 850° C.

6. The composite of claim 5, wherein the low melting glass-ceramic material is thickened by dissolving the pyrogenic silica.

7. The composite of claim 1, wherein the facing material is curable using light-, electron beam-, moisture-, heat-or self-curing methods.

8. The composite of claim 1, wherein the facing material is at least partially translucent.

9. The composite of claim 1, wherein the sintering is controlled by added melt modifiers including a salt such as at least one of: Aluminum-, Calcium-, Magnesium-, or Boron salts.

10. The composite of claim 1, wherein the facing material is packaged in bottles, syringes, as a flat sheet and/or a strip condition.

11. A ceramic dental prosthetic comprising:
a ceramic base;
a moldable and three-dimensionally stable facing material molded to surfaces of the base, polymerized, and sintered onto the base to provide the look of natural surfaces.

12. The ceramic dental prosthetic of claim 11, wherein the facing material is formed at least in part from a polymeric material.

13. The ceramic dental prosthetic of claim 11, wherein the facing material is light-, electron beam-, moisture-, heat-or self-pre-curable.

14. The ceramic dental prosthetic of claim 11, wherein the facing material is at least partially translucent.

15. The ceramic dental prosthetic of claim 11, wherein the facing material is sintered between 800° C. and 1550° C.

16. The ceramic dental prosthetic of claim 15, wherein the facing material is sintered to the already sintered ceramic.

17. The ceramic dental prosthetic of claim 11, wherein the facing material maintains its color during preparation, polymerization and sintering.

18. The ceramic dental prosthetic of claim 11, further comprising colored ceramic fibers for replicating a vein structure of gingival tissue.

19. The ceramic dental prosthetic of claim 11, further comprising liquid ceramic modifiers to provide characterization to the facing material-modified base.

20. A method of adding esthetic features to a ceramic dental prosthetic base, the method comprising:
applying a moldable and three-dimensionally stable facing material to surfaces of the base;
trimming the facing material to conform to the surfaces of the base;
curing the facing material into a hardened condition for evaluation, and
sintering the facing material into a ceramic state.

21. The method of claim 20, wherein the facing material is formed at least in part from a polymeric material.

22. The method of claim 20, wherein the facing material is formed at least in part from a Glass-Ceramic material.

23. The method of claim 20, further comprising using a pyrogenic silica incorporated in the facing material to jellify the liquid glass melt to maintain the shape during sintering.

24. The method of claim 20, wherein the step of curing the facing material further comprises curing the facing material using light, electron beam, moisture, heat or self-curing.

25. The method of claim 20, wherein the final step of applying the facing material further comprises sintering the facing material using ceramic sintering ovens.

26. The method of claim 20, further comprising using ceramic fibers to replicate a vein structure of gingival tissue.

27. The method of claim 20, further comprising using liquid modifiers to provide characterization to the facing material-modified base.
